(19) United States
(12) Patent Application Publication
(10) Pub. No.: US 2025/0064678 A1
(43) Pub. Date: Feb. 27, 2025

(54) FLUID DISPENSING DEVICE AND FLUID CONTAINER

(71) Applicant: A. NATTERMANN & CIE GMBH, Frankfurt am Main (DE)

(72) Inventors: Sabine GOTTKE, Lohne (DE); Thomas HINNEN, Wuppertal (DE)

(21) Appl. No.: 18/720,317

(22) PCT Filed: Dec. 14, 2021
(86) PCT No.: PCT/IB2021/061697

§ 371 Date: Jun. 14, 2024

(51) Int. Cl. A61J 1/14; A61M 15/08

(52) U.S. Cl. CPC A61J 1/14; A61M 15/08

(57) ABSTRACT

The present disclosure relates to a fluid dispensing device (10) and to a fluid container (110) for such a dispensing device (1), the fluid container (110) comprising:—a flexible bag (120) comprising a flexible sidewall (122), the flexible sidewall (122) extends along a longitudinal direction (z), confines an interior volume (123) to receive the fluid and forms a bag outlet (124) towards a distal end,—a rigid fastening adapter (112) sealingly engaged with the bag outlet (124) and comprising a hollow outlet shaft (113) in fluid communication with the interior volume (123),—wherein the rigid fastening adapter (112) comprises a fastening structure (114) for mechanical engagement with a corresponding counter fastening structure (134) of the fluid dispensing device (1) to establish a fluid-tight connection between the fluid discharge mechanism (130) and the outlet shaft (113).

### TECHNICAL FIELD

The present disclosure relates to the field of fluid dispensing devices and in particular to fluid dispensing devices configured as nasal inhalers. The disclosure further relates to spray devices configured to dispense a fluid or a liquid substance by way of spraying or atomizing. The disclosure also relates to a mechanical energy storage for use in a fluid dispensing device, e.g. for driving a discharge mechanism of the fluid dispensing device.

### BACKGROUND

Fluid dispensing devices operable to atomize a liquid substance are as such known. Such devices typically comprise an outlet orifice, e.g. integrated in or provided by a nozzle. Upon application of a force by a user to an actuation lever or a button the fluid is dispensed via the outlet orifice. Such devices may be arranged to dispense a single or multiple doses and may be equipped with a container providing a reservoir for the fluid thus allowing and supporting the dispensing of several doses.

Such fluid dispensing devices may be provided with a mechanical energy storage operable to provide a force effect for discharging and/or atomizing of the fluid. Here, a user may induce a spray dispensing of the fluid by depressing a trigger by way of which mechanical energy provided by the mechanical energy storage is released for the fluid dispensing.

Charging or preloading of the mechanical energy storage may be induced by user interaction. With existing fluid dispensing devices a user has to recharge or preload the mechanical energy storage every time a fluid dispensing action has been triggered.

It is generally desirable to improve operability and user handling of such fluid dispensing devices. Moreover, user acceptance of such fluid dispensing device should be enhanced. It is also desirable to provide improve solutions for a fluid container operable with fluid dispensing devices, such as nasal inhalers.

### SUMMARY

In one aspect the present disclosure relates to a fluid container for a fluid dispensing device. The fluid dispensing device comprises a fluid discharge mechanism. Typically, the fluid discharge mechanism is operable for spray discharging multiple doses of the fluid via an outlet orifice of the fluid dispensing device.

With some examples the fluid dispensing device is implemented as a nasal inhaler. The outlet orifice may be provided on or integrated into a nozzle. The outlet orifice may be provided at a free end or distal end of a tapered nozzle. Such a nozzle may be configured and shaped for insertion into a nostril of a user. The outlet orifice may be configured to atomize the fluid, e.g. by way of an atomizer provided in or on the outlet orifice.

The fluid container comprises a flexible bag comprising a flexible side wall. With some examples the flexible bag may be constituted by the flexible side wall. The flexible sidewall and hence the flexible bag extends along a longitudinal direction. The flexible bag confines an interior volume to receive and/or to accommodate the fluid. The flexible bag further comprises a bag outlet towards a longitudinal distal end. The bag outlet may be formed by the flexible sidewall. It may be provided at a longitudinal end of the flexible sidewall.

The fluid container further comprises a rigid fastening adapter sealingly engaged with the bag outlet and comprising a hollow outlet shaft in fluid communication with the interior volume. The rigid fastening adapter comprises a fastening structure for mechanical engagement with a corresponding counter fastening structure of the fluid dispensing device. The rigid fastening adapter is configured and operable to establish a fluid tight connection between the fluid discharge mechanism of the fluid dispensing device and the outlet shaft of the fluid container.

Hence, when the fastening structure of the fluid container is mechanically engaged with the corresponding or complementary-shaped counter fastening structure of the fluid dispensing device the fluid discharge mechanism of the fluid dispensing device is e.g. in fluid tight and hence liquid proof connection with the outlet shaft of the fluid container.

Providing a fluid container with a flexible bag and with a rigid fastening adapter provides the benefit to enable a rather smooth and easy withdrawal of the fluid from the interior volume via the outlet shaft by way of suction. By withdrawing an amount of the fluid from and through the bag outlet the flexible bag may become subject to a respective deformation, in particular of its flexible side wall. Hence, the flexible bag and its flexible sidewall are configured to adapt to the reduced interior volume as an amount of the fluid is withdrawn from the flexible bag.

The flexible bag and/or the flexible sidewall do not generate any or at most negligible restoration forces to return the flexible back in an initial configuration. Insofar, the flexible bag only imparts a rather low or negligible resistance against withdrawal of the fluid from the interior volume of the flexible bag.

The fluid tight fastening of the bag outlet with the rigid fastening adapter and the fluid communication with the hollow shaft provides the benefit to enable a rather precise, long-lasting, liquid-proof, and well-defined mechanical connection between the fluid container and the fluid discharge mechanism of the fluid dispensing device.

The rigid fastening adapter provides an easy and reliable mutual assembly and a rather fluid-tight connection between the flexible bag and the fluid discharge mechanism. Moreover, by way of the rigid fastening adapter the fluid container can be handled in a well-defined way not only for use with the fluid dispensing device but also for transportation and storage. The rigid fastening adapter may provide a handle and/or a well-defined mechanical handling of the fluid container during transportation, storage, and assembly with the fluid dispensing device.

According to a further example the hollow outlet shaft protrudes outwardly from the interior volume as confined by the flexible bag. The outwardly protruding shaft is of particular benefit so as to facilitate fastening of the fluid container in the fluid dispensing device and for establishing a fluid tight interconnection with the fluid discharge mechanism of the fluid dispensing device.

Typically, the fluid discharge mechanism comprises a recess sized and shaped to sealingly engage with the hollow outlet shaft of the fluid container. According to a further example the rigid fastening adapter comprises a shoulder portion adjacent to the distal end of the flexible bag. Here, the fastening structure is provided on at least one of an outside surface of the shoulder portion and an outside surface of the outlet shaft. The shoulder portion may comprise a somewhat planar-shaped or flange-like disc or board effectively closing the bag outlet.

By providing the fastening structure on at least one of an outside surface of the shoulder portion and an outside surface of the outlet shaft respective fastening structures are easily accessible from outside the fluid container thus allowing for a rather easy and straightforward assembly with a complementary-shaped counter fastening structure of the fluid dispensing device.

According to another example the hollow outlet shaft of the fluid container protrudes from the shoulder portion in longitudinal distal direction. The shoulder portion may further comprise a through recess merging with an interior of the hollow outlet shaft. Insofar, the shoulder portion forms a kind of a narrowing shoulder of the fluid container.

With some examples the shoulder portion comprises a disc or board with an outer edge that coincides with a respective longitudinal edge of the bag outlet. The bag outlet, e.g. a distal end of the sidewall may be sealed with the edge of the disc or board of the shoulder portion so as to provide a fluid-tight engagement between the shoulder portion and the bag outlet. The outlet shaft may be integrally formed with the shoulder portion. A hollow interior of the outlet shaft may penetrate the shoulder portion, e.g. a central portion thereof. With other examples the shoulder portion only serves to provide a longitudinal abutment for the bag. The through recess extending through the shoulder portion and merging with the interior of the hollow outlet shaft may receive the bag outlet, wherein the bag outlet may be located and arranged inside the hollow outlet shaft of the fluid container.

According to a further example the shoulder portion comprises a distally facing abutment face to longitudinally abut with the counter fastening structure of the fluid dispensing device. In this way the shoulder portion may provide a longitudinal stop portion delimiting an insert motion of the shoulder portion into a complementary-shaped receptacle of the fluid dispensing device. With some examples the counter fastening structure of the fluid dispensing device may get in longitudinal abutment with the distally facing abutment face of the shoulder portion, thereby defining an end stop for inserting the shoulder portion into the complementary-shaped receptacle of the fluid dispensing device.

According to a further example the fastening structure comprises at least one snap element to mechanically engage in a form-fitting or positively engaging manner with a complementary-shaped counter snap element of the counter fastening structure of the fluid dispensing device. A snap element or numerous snap elements provided on the rigid fastening adapter to engage with complementary-shaped snap elements of the counter fastening structure provide a rather easy and straightforward mutual assembly and fastening of the container inside the housing of the fluid dispensing device thereby inherently providing a liquid-proof or fluid-tight engagement between the hollow outlet shaft of the rigid fastening adapter and the fluid discharge mechanism of the fluid dispensing device.

Implementing a snap element to engage with a counter snap element is also beneficial to provide an audible feedback when the respective snap element(s) engage(s) with the counter snap element(s). This way, and upon a final assembly of the fluid dispensing device and upon inserting the fluid container into the fluid dispensing device there can be provided a well-defined acoustic or haptic feedback indicating that a fluid tight connection is established between the fluid discharge mechanism at the outlet shaft of the fluid container.

According to another example the snap element comprises a beveled flank or edge adjoining a proximally facing abutment face. The counter snap element of the counter fastening structure typically comprises a complementary-shaped beveled flank or edge adjoining a distally facing abutment face. In the process of mutual assembly, the beveled flank of the snap element glides or slides along the complementary shaped beveled flank of the counter snap element until the proximally facing abutment face of the snap element engages the distally facing abutment face of the counter snap element.

Typically, the snap element and the complementary-shaped counter snap element are configured or shaped as a latch, as a catch and/or as a barb, which when mutually engaged provide a fixing of the fastening structure to the counter fastening structure with regards to the longitudinal direction (z).

According to a further example the fluid container comprises an outer rigid casing sized to accommodate the flexible bag. The outer rigid casing is fixedly connected to the rigid fastening adapter. Alternatively or additionally, the rigid fastening adapter is integrated into the outer rigid casing. The outer rigid casing may provide a twofold function. First of all, it may provide the rigid fastening adapter. Second, it may completely enclose the flexible bag. This way, the flexible bag can be protected by the outer rigid casing against environmental influences.

Moreover, by entirely enclosing the flexible bag by the outer rigid casing the flexible bag can be also protected against electromagnetic radiation, such UV radiation and the like environmental influences that may harm the integrity or efficacy of the fluid located inside the fluid container.

According to a further example the outer rigid casing is made of a material or a material composition comprising at least one of a high density polyethylene and a polypropylene. With some examples the outer rigid casing may comprise numerous homogeneous layers of a high density polyethylene and a polypropylene. Such a combination of plastic materials is beneficial to provide a rigid casing in order to protect the flexible bag located inside.

According to a further example the outer rigid casing comprises a multilayer structure with a first layer made of a high density polyethylene a second layer made of a polypropylene. The first layer is located outside the second layer. This way, the respective properties of both, high-density polyethylene and polypropylene can be combined for providing and forming the outer rigid casing. Both, polypropylene as well as high-density polyethylene are highly stable against chemical aggressive substances, such as acids or alkalis.

According to a further example the flexible bag is blow molded or injection molded into the outer rigid casing. Alternatively, the flexible bag and the outer rigid casing are co-extruded. With both examples, a rather elegant approach for providing a flexible bag in an outer rigid casing are provided that are particularly applicable for mass manufacturing. In effect, by blow molding or injection molding the flexible bag inside the outer rigid casing forms a kind of a “bag in a bottle”-solution. Here, the flexible bag, in particular its bag outlet can be fixedly attached to the outer rigid casing as such or to the rigid fastening adapter.

According to a further example the bag outlet of the flexible bag is sealingly engaged with the rigid fastening adapter through or by at least one of a clamping, a bonding, a welding, an extrusion, a blow molding, or an injection molding. Other portions of the flexible bag may be only loosely fitted or located inside the interior space as provided by the rigid fastening adapter or outer rigid casing. This enables collapsing of the flexible bag upon withdrawal of the fluid through the outlet shaft.

According to a further example an outside surface section of the bag outlet is sealingly engaged with an inside surface of the hollow outlet shaft. This way, it is exclusively an outside surface of the flexible bag that is fastened or sealingly engaged with the rigid fastening adapter, e.g. by way of one of clamping, bonding, welding, extruding, blow molding or injection molding. In this way, an inside surface of the bag outlet and hence an inside of the bag may not be subject to application of heat required for a sealing engagement between the bag outlet and the rigid fastening adapter. This is of particular benefit for keeping the inside surface of the flexible bag substantially inert with regards to the fluid contained inside the flexible bag.

According to a further example the hollow outlet shaft of the fluid container comprises a cylindrical receptacle at a distal end to receive a complementary shaped valve insert of the fluid discharge mechanism. The cylindrical receptacle of the hollow outlet shaft provides a well-defined mechanical and fluid tight interface of the fluid container for enabling a rather precise, well-defined, and liquid proof fluid transferring interconnection between the fluid container and the fluid discharge mechanism.

According to a further example of the fluid container the hollow outlet shaft comprises a tapered seal seat section to sealingly engage with the valve insert. Typically, the valve insert comprises a complementary shaped tapered counter seal seat section. This way and upon inserting the valve insert into the hollow outlet shaft there may be established a liquid-proof and fluid transferring seal seat between the outlet shaft and the valve insert, thus providing a liquid proof transportation of the fluid from the interior of the fluid container towards and into the fluid discharge mechanism of the fluid dispensing device.

According to a further example the flexible bag is made of a material or material composition comprising at least one of an ethylene vinyl alcohol, a low density polyethylene and a high density polyethylene. The combination of these material components provides a rather flexible and liquid proof, hence impenetrable side wall of the flexible bag.

According to a further example the flexible bag comprises a multilayer structure with a first layer of an ethylene vinyl alcohol and a second layer made of at least one of a low density polyethylene and a high density polyethylene. With some examples the flexible bag, e.g. the sidewall of the flexible bag comprises three individual layers of respective plastic components.

The first layer may be made of an ethylene vinyl alcohol, the second layer may comprise a low density polyethylene, optionally including an adhesive, and the third layer may comprise at least one of a low density polyethylene and a high density polyethylene. The first layer is located outside the second layer and the second layer is located outside the third layer. Such a layer combination is of particular use to provide a liquid proof and hence impenetrable flexible bag exhibiting a comparatively low degree of mechanical stiffness and thus allowing to withdraw an amount of the fluid by way of suction without generating a respective back pressure.

According to another aspect the present disclosure also relates to a fluid dispensing device. The fluid dispensing device comprises a housing to accommodate a fluid container as described above. The fluid dispensing device also comprises an outlet orifice for spray discharging an amount of the fluid as provided by the fluid container. The fluid dispensing device further comprises a mechanical energy storage coupled to the discharge mechanism, reversibly transferable between a preloaded state and an unloaded state.

The mechanical energy storage is further configured to store mechanical energy in the preloaded state, which mechanical energy effective to produce a spray discharging of the fluid the outlet orifice. The discharge mechanism is operable for spray discharging multiple doses of the fluid via the outlet orifice by way of mechanical energy released from the mechanical energy storage.

The fluid dispensing device also comprises a counter fastening structure to mechanically engage with the fastening structure of the rigid fastening adapter of the fluid container as described above. This way, the counter fastening structure is configured to establish the fluid tight connection between the fluid discharge mechanism and the outlet shaft of the fluid container.

The fluid dispensing device is particularly adapted to cooperate and to interact with the fluid container as described above. Insofar, all features, effects, and benefits as described above in connection with the fluid container equally apply to the fluid dispensing device; and vice versa.

Generally and as used herein, a preloaded state is a state, in which the mechanical engineering storage stores at least a non-zero portion of mechanical energy. Insofar the preloaded state is a loaded state. The term “preloaded” as used herein may further indicate and/or imply, that the fluid dispensing device can be stored in a loaded state, e.g. over a comparatively long time interval. Then and while not in use the fluid dispensing device is and remains mechanically biased and is immediately ready to use for discharging a dose of the fluid. Typically, preloading of the mechanical energy storage may be provided at the end of a dose dispensing procedure.

According to a further example the discharge mechanism of the fluid dispensing device comprises a driver operatively connected to or coupled to the mechanical energy storage and movable relative to one of the housing and the outlet orifice to effectuate a spray discharging of the fluid. The driver further comprises the counter fastening structure to mechanically engage with the rigid fastening adapter of the fluid container.

The driver may be implemented as a kind of a chassis. With some examples the driver forms an inner housing sized to accommodate the entirety of the fluid container. This way, the driver and the fluid container may form or constitute a container pre-assembly configured for arrangement and/or fastening inside the housing of the fluid dispensing device.

Typically and with some examples, the driver is longitudinally slidably guided in or on the housing along a longitudinal direction between a biased position and an unbiased position. The driver is movable into the biased position by user interaction, e.g. by a biasing mechanism of the fluid dispensing device. By moving the driver into the biased position mechanical energy is transferred to the mechanical energy storage in order to bring the mechanical energy storage into the preloaded state. Typically, the fluid dispensing device further comprises a releasable interlock configured to retain the mechanical energy storage in the preloaded date.

The fluid dispensing device may be further equipped with a trigger mechanism operably engaged or coupled with the interlock and operable to release mechanical energy stored in the mechanical energy storage when actuated. Actuation of the trigger mechanism at least temporally suspends the releasable interlock and allows the driver to be moved from the biased position towards the unbiased position. Movement of the driver towards the unbiased position serves to generate a spray discharging of a dose of the fluid by the outlet orifice.

According to a further example the discharge mechanism comprises an inlet valve in fluid connection with the outlet shaft of the fluid container when the fluid container is properly assembled and arranged inside the fluid dispensing device. The discharge mechanism further comprises an outlet valve in fluid connection with the outlet orifice. The inlet valve may be provided by or implemented in the valve insert as described above. The discharge mechanism also comprises a dispensing chamber located between the inlet valve and the outlet valve. The dispensing chamber is sized to receive at least one dose of the fluid.

With some examples the dispensing chamber is sized to receive numerous doses of the fluid. Here, the driver is movable from the biased position towards and into the biased position in numerous discrete steps, wherein each stepped movement of the driver from the biased position towards and into the unbiased position serves to expel a single dose of the fluid.

Moreover and for effectuating the spray discharging of the fluid, a size of the dispensing chamber is reducible by moving at least one of the inlet valve and the outlet valve relative to the other one of the inlet valve and the outlet valve. With some examples the outlet valve is fixed with regards to the outlet orifice and the inlet valve is movable relative to the outlet valve so as to increase the dispensing chamber, e.g. during a suction phase of the discharge mechanism.

During an expelling phase, the inlet valve is movable towards the outlet valve, thereby reducing the size of the dispensing chamber in such a way that the fluid located inside the dispensing chamber is expelled via the outlet valve. Typically, the inlet valve and/or the outlet valve are implemented as a check valve and/or as a one-way valve. The inlet valve is operable to allow a fluid flow into the dispensing chamber but serves to prevent an escape of the fluid from the dispensing chamber. The outlet valve is operable to prevent ingress of air or of a liquid substance into the dispensing chamber and is exclusively operable to expel the fluid from the dispensing chamber.

According to a further example the fluid dispensing device comprises the container as described above, which is filled with the fluid and which is connected to the outlet orifice in a fluid transferring manner. Typically, the container is assembled inside the housing of the fluid dispensing device and is in liquid proof or fluid transferring connection with the discharge mechanism of the fluid dispensing device.

With some examples the container may be releasably attachable to the discharge mechanism. The container may be arranged in a removable manner inside the housing of the fluid dispensing device. Hence, the fluid dispensing device may be implemented as a reusable device offering to replace the container when empty. With other examples the fluid dispensing device is implemented as a disposable device. Here, the container filled with the fluid may be permanently located inside the housing of the fluid dispensing device. Then, the container may not be exchangeable arranged inside the housing. When the container is empty the entire fluid dispensing device may be intended to become discarded.

With some examples the mechanical energy storage comprises at least a first drive spring which is resiliently compressible in the longitudinal direction (z) to store mechanical energy. The drive spring is operable to induce a longitudinal motion of the driver when the interlock is released by actuation of the trigger. With some examples the mechanical energy storage comprises a first drive spring and a second drive spring. The first and the second drive springs may be oriented parallel to each other and may be arranged on opposite sides of the driver. This way, a rather smooth and tilt free longitudinal sliding displacement of the driver relative to the housing can be provided.

Generally, the scope of the present disclosure is defined by the content of the claims. The fluid dispensing device and the fluid container is/are not limited to specific embodiments or examples but comprise(s) any combination of elements of different embodiments or examples. Insofar, the present disclosure covers any combination of claims and any technically feasible combination of the features disclosed in connection with different examples or embodiments.

In the present context the term “distal” or “distal end” relates to an end of the fluid container or fluid dispensing device that faces towards an application site of a person or of an animal. The term ‘proximal’ or ‘proximal end’ relates to an opposite end of the injection device, which is furthest away from an application site of a person or of an animal.

The terms “fluid”, “drug” or “medicament” are used synonymously herein and may describe a at least one of consumer health care product and a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders. A consumer health care product may be void of an active pharmaceutical ingredient. It may be commercially available free of prescription. As a nonlimiting examples consumer health care products may include products such as nasal sprays, cough syrups, eyedrops, creams, ointments, dietary and nutrition supplements and/or cosmetics.

As described below, a fluid, drug or medicament can include at least one API, or combinations thereof, in various types of formulations, for the treatment of one or more diseases. Examples of API may include small molecules having a molecular weight of 500 Da or less; polypeptides, peptides, and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (SIRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days, alternatively 1 to at least 10, 15, 20, or 25 days). In some instances, the chamber may be designed to store a drug for about 1 month to about 2 years, alternatively from about 1 month to about 6 months, alternatively from about 1 month to about a year, alternatively from about 1 month to 1.5 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders.

Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys (B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des (B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems, and embodiments described herein may be made without departing from the full scope and spirit of the present invention, which encompass such modifications and any and all equivalents thereof.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE DRAWINGS

The details of particular implementations are set forth in the accompanying drawings and description below. Like reference numerals may refer to like elements throughout the specification. Other features will be apparent from the following description, including the drawings and claims. The drawings, though, are for the purposes of illustration and description only and are not intended as a definition of the limits of the disclosure.

In the following, numerous examples of a fluid dispensing device will be described in greater detail by making reference to the drawings, in which:

FIG. 1 shows an example of a fluid dispensing device implemented as a spray delivery device,

FIG. 2 shows the device in a configuration, wherein a protective cap is in an open position,

FIG. 3 shows the device in the course of dispensing a dose of the fluid,

FIG. 4 shows a perspective illustration of individual components of the fluid dispensing device,

FIG. 5 shows a perspective illustration of a closure of the fluid dispensing device,

FIG. 6 is a longitudinal cross-section through the closure of FIG. 5,

FIG. 7 shows a detail of the closure provided with a trigger mechanism,

FIG. 8 shows a cross-section through the arrangement of FIG. 7,

FIG. 9 shows the trigger mechanism in an initial configuration,

FIG. 10 shows the trigger mechanism when a trigger button is depressed for a first time,

FIG. 11 shows the trigger mechanism after depressing the trigger button for a first time,

FIG. 12 shows the trigger mechanism after release of the trigger button,

FIG. 13 shows a longitudinal cross-section through the fluid dispensing device,

FIG. 14 shows a cross-section through a discharge mechanism before dispensing of a first dose of the fluid,

FIG. 15 shows the discharge mechanism after dispensing of a first dose of the fluid,

FIG. 16 shows the discharge mechanism after dispensing of a second dose and

FIG. 17 shows the discharge mechanism after dispensing of a third dose,

FIG. 18 shows an example of a driving spring arrangement of a mechanical energy storage,

FIG. 19 shows another example of two driving springs,

FIG. 20 shows an example, wherein the drive springs are constituted by numerous spring elements,

FIG. 21 shows an initial configuration of a mechanical energy storage in an initial configuration, wherein the protective cap of the fluid dispensing device is in a closed position,

FIG. 22 shows a detail of the configuration according to FIG. 21,

FIG. 23 is illustrative of a configuration of the mechanical energy storage after opening of the protective cap,

FIG. 24 shows the mechanical energy storage of the dispensing of a first dose of the fluid,

FIG. 25 is illustrative of the mechanical energy storage after dispensing of a second dose of the fluid,

FIG. 26 shows a configuration of the mechanical energy storage after dispensing of a third those of the fluid,

FIG. 27 shows a cross-section through an example of a fluid container of the fluid dispensing device,

FIG. 28 shows the fluid container configured for mechanical engagement with the fluid discharge mechanism of the fluid dispensing device,

FIG. 29 shows a further example of a fluid container,

FIG. 30 shows a cross-section through the fluid container according to FIG. 29,

FIG. 31 shows a transverse cross-section through the fluid dispensing device,

FIG. 32 shows a proximal end of a driver of the fluid dispensing device,

FIG. 33 is an enlarged view of the interaction between the protective cap and a biasing mechanism in a perspective illustration,

FIG. 34 is an enlarged view of a cross-section through a biasing mechanism configured for storing mechanical energy in the mechanical energy storage,

FIG. 35 shows a mutual position of a biasing member relative to a driver of the discharge mechanism with the driver in the biased position,

FIG. 36 shows the arrangement of FIG. 35, wherein the driver is in the unbiased position,

FIG. 37 shows the biasing mechanism, wherein the protective cap is in an open position,

FIG. 38 shows the biasing mechanism, wherein the protective cap is in an intermediate position,

FIG. 39 shows the biasing mechanism with the protective cap in a closed position,

FIG. 40 shows the housing of the fluid dispensing device and the protective cap in a disassembled configuration,

FIG. 41 shows the housing and the protective cap when mutually assembled, and

FIG. 42 shows a cross-section through a hinge by way of which the protective cap is pivotably lease supported on the housing.

### DETAILED DESCRIPTION

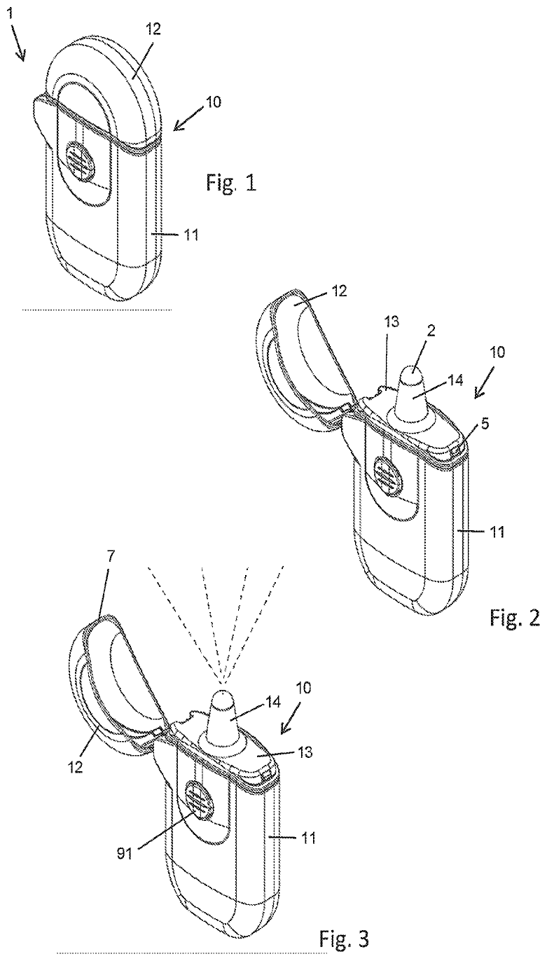

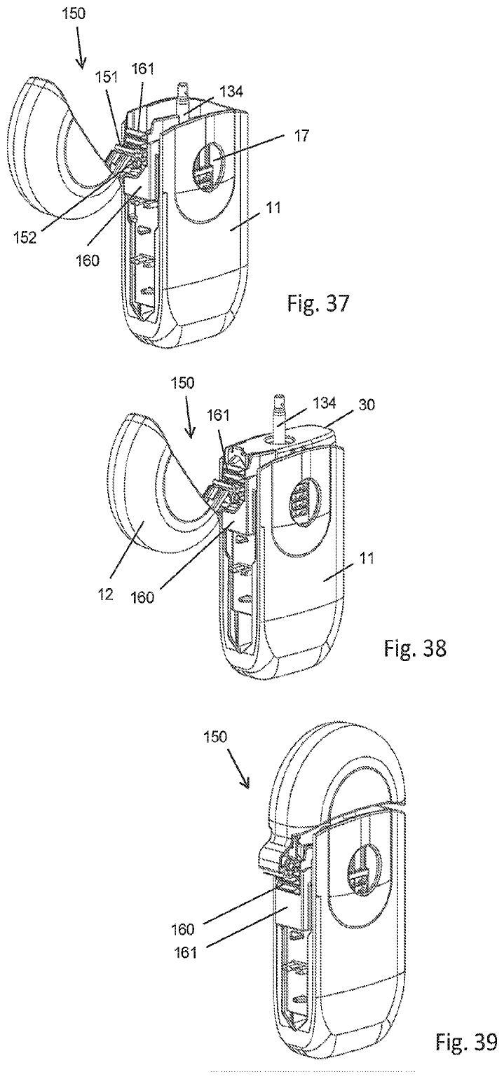

In FIGS. 1-42 numerous examples and configurations of a fluid dispensing device 1 are schematically illustrated. The fluid dispensing device 1 may be implemented as a nasal inhaler. The fluid dispensing device 1 comprises a housing 10. The housing 10 comprises a body 11 sized to accommodate a fluid container 110 filled with a dispensable fluid. The fluid may comprise a medicament comprising a pharmaceutically active substance.

The fluid container 110 may be entirely arranged inside the hollow and rather cup-shaped body 11 of the housing 10. The housing 10 further comprises a protective cap 12. The protective cap 12 is sized and shaped to enclose an outlet orifice 3, e.g. provided at or in a nozzle 14. The nozzle 14 may comprise a conically-shaped protrusion sized for insertion into a nostril of a user. A distal end of the nozzle 14 may be provided with the outlet orifice 3. The outlet orifice 3 may be part of an atomizer 2 configured and shaped to atomize or to nebulize the fluid when dispensed by or through the nozzle 14.

The nozzle 14 may be implemented as a part of a closure 13 configured for fastening to a distal end of the cup-shaped body 11. The closure 13 may be clipp-fastened to the upper or distal end of the body 11. The closure 13 may be detachably or undetachably connected to the sidewall 18 of the body 11.

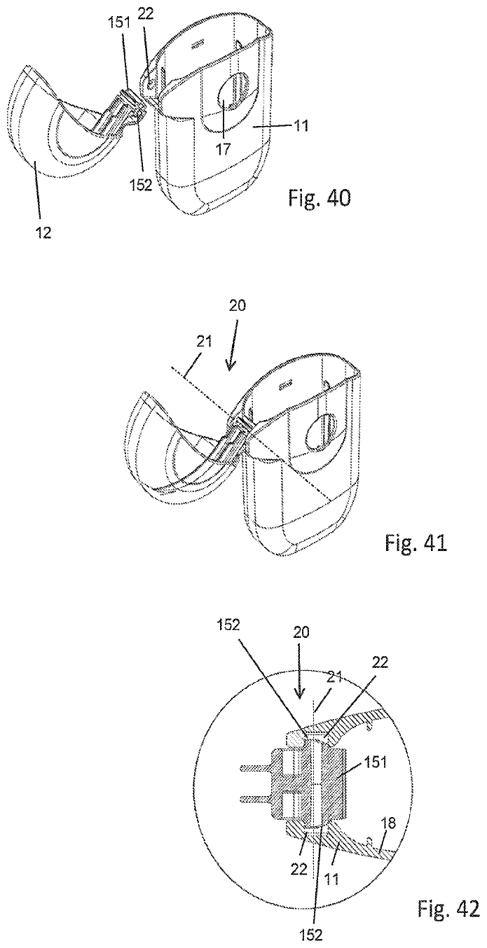

The protective cap 12 is pivotally supported or arranged on the housing 11. It may be hingedly attached to the housing 11 by way of a hinge 20. For this, the protective cap 12 comprises a hinge axis 21. The housing 11 comprises two oppositely located recesses 22 sized and shaped to receive an axial protrusion 152 of a pinion segment 151 integrally formed or attached to the protective cap 12 as illustrated in FIGS. 40-42.

The axial protrusions 152 may be snap fitted into the oppositely located recesses 22 and may be pivotally supported in the recesses 22 on the inside surface of the sidewall 18. This way, the protective cap 12 can be pivoted relative to the body 11 between a closed position as illustrated in FIG. 1 and an open position as illustrated in FIG. 3.

Inside the fluid dispensing device 1 there is provided a driver 30, which is longitudinally displaceable relative to the housing 10 along a longitudinal direction (z). The driver 30 is implemented as a part of a discharge mechanism 130. The discharge mechanism 130 comprises or forms a pump by way of which one or several doses of the fluid can be extracted or withdrawn from the fluid container 110 and by way of which one or multiple doses of the fluid can be expelled through the nozzle 14 by one or several spray delivery actions.

The driver 30 and hence the discharge mechanism 130 is biased by a mechanical energy storage 50. The mechanical energy storage 50 comprises at least one drive spring 51, 52 by way of which mechanical energy can be stored in the fluid dispensing device 1. The mechanical energy storage 50 is operatively coupled or engaged with the discharge mechanism 130 and is transferable between a preloaded state and an unloaded state. The mechanical energy storage 50 is configured to store mechanical energy in the preloaded state, which mechanical energy is effective and sufficient to produce the spray discharging of the fluid.

The fluid dispensing device 1 further comprises a releasable interlock 70, which is configured to retain the mechanical energy storage 50 in the preloaded state. The fluid dispensing device 1 further comprises a trigger mechanism 90 operably engageable or operably engaged with the interlock 70. When engaged with the trigger mechanism 90 the interlock is operable to release at least a first portion of the mechanical energy stored in the mechanical energy storage when actuated for a first time. The trigger mechanism 90 is further operable to release at least a second portion of the mechanical energy stored in the mechanical energy storage 50 when actuated for a second time.

In other words, the mechanical energy storage 50, the releasable interlock 70 and the trigger mechanism 90 are configured to provide numerous, i.e. at least a first and a second spray delivery or spray discharging of the fluid upon repeated actuation of the trigger mechanism without an intermediate preloading or re-charging of the mechanical energy storage 50.

As will be described below in greater detail the mechanical energy storage 50 can be preloaded or charged by transferring the protective cap 12 from the open position as illustrated in FIG. 3 into the closed position as illustrated in FIG. 1. With other examples of the fluid dispensing device 1 it is also conceivable to charge or to preload the mechanical energy storage 50 when transferring the protective cap 12 from the closed position as illustrated in FIG. 1 into the open position as e.g. illustrated in FIG. 3. With any example it is intended that a user provides a respective torque or pivoting of the protective cap 12 sufficient to charge or to preload the mechanical energy storage 50.

With the presently illustrated example it is intended that the mechanical energy storage 50 is preloaded or charged with mechanical energy upon and by transferring the protective cap 12 from the open position into the closed position. This way it can be ensured, that the mechanical energy storage is sufficiently charged or preloaded since the closing action of the protective cap 12 is easily controllable by the end user and is inherently provided with a mechanical, haptic, and e.g. audible feedback, e.g. when a snap feature 5 as provided on one of the body 11 or a closure 13 engages with a complementary shaped counter snap feature 7 as provided on the protective cap 12.

Dispensing of a dose of the fluid contained inside the fluid container 110 is provided by moving the driver 30 relative to the nozzle 14. Since the nozzle 14 is rigidly connected or fixed to the body 11 delivery or dispensing of a dose of the fluid is also provided by moving the driver 30 relative to the housing 10 or relative to the body 11 along the longitudinal direction (z).

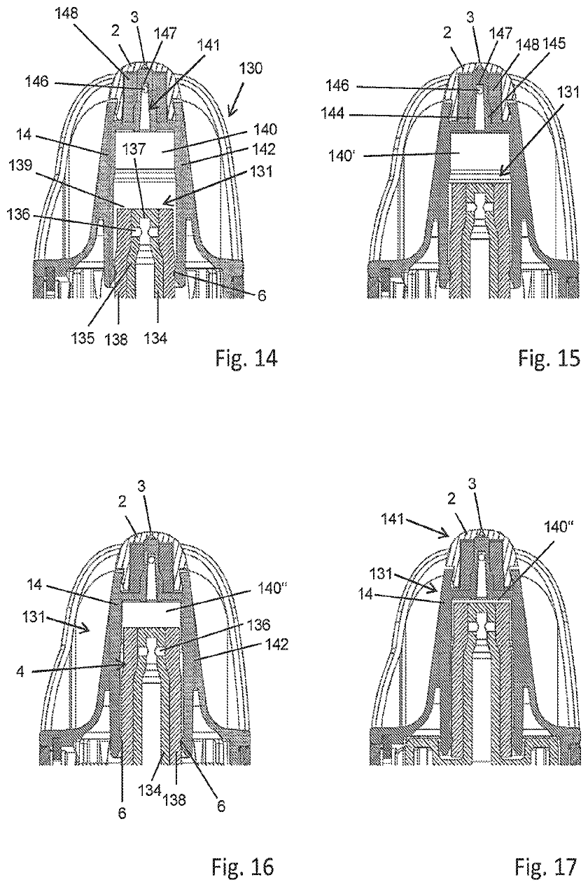

With the presently illustrated example a dose of the fluid is dispensed by moving the driver 30 relative to the housing 10 in longitudinal distal direction, hence towards the nozzle 14. The driver 30 is part of a discharge mechanism 130. The discharge mechanism 130 comprises a kind of a pump mechanism. The discharge mechanism 130 comprises an inlet valve 131 and an outlet valve 141 as illustrated in FIG. 14. The inlet also 131 and the outlet valve 141 may be both implemented as a check valve or as a one way valve. The inlet valve 131 is sealingly engaged with a dispensing chamber 140. The inlet valve 131 is provided upstream of the dispensing chamber 140. The outlet valve 141 is provided downstream of the dispensing chamber 140.

The dispensing chamber 140 comprises a tubular sidewall 142 provided inside the nozzle 14 as illustrated in the sequence of FIGS. 14-17. A proximal end of the tubular sidewall 142 is sealingly engaged with the inlet valve 131. A distal end of the dispensing chamber 140 is sealingly engaged with the outlet valve 141. The inlet valve 131 comprises an inner tubular section 134 comprising a tubular-shaped sidewall 135. The hollow interior of the inner tubular section 134 is in permanent flow connection with the interior of the fluid container 110.

An outside surface of the inner tubular section 134 is sealed by a tubular sheath 138 of a flexible material. The tubular sheath 138 may comprise a polymeric or elastomeric material being elastically deformable. The inner tubular section 134 and hence its hollow interior is confined in distal direction by a closed end face 137. At a predefined distance from the distal end face 137 the sidewall 135 comprises at least one through opening 136. The through opening 136 or several through openings is/are a radially covered and sealed by the tubular sheath 138. A distal end face 139 of the tubular sheath 138 is flush with a respective outer end face of the inner tubular section 134.

Towards a proximal end, the dispensing chamber 140 is sealingly engaged with an outside surface of the tubular sheath 138. Here, an inside surface of the sidewall 142 is provided with a sealing lip 6. The sealing lip 6 may comprise an annular protrusion and may be in fluid-tight but longitudinally smoothly movable engagement with the outside surface of the tubular sheath 138. The tubular sheath 138 is tightly fitted to the outside surface of the inner tubular section 134. In situations, wherein a fluid pressure inside the dispensing chamber 140 is lower than a fluid pressure inside the inner tubular section 134 the fluid provided inside the hollow part of the inner tubular section 134 is sucked or drawn into the dispensing chamber 140.

Here, a pressure gradient between the dispensing chamber 140 and the hollow interior of the inner tubular section 134 serves to urge the fluid through the at least one through opening 136 into a slot or gap formed between the inside surface of the tubular sheath 138 and the outside surface of the inner tubular section 134. Due to the pressure gradient the distal end of the tubular sheath 138 may widen in radial direction so as to form a respective slot, gap, or slit and to enable a transfer of the fluid from the interior of the inner tubular section 134 into the dispensing chamber 140.

The distal end of the dispensing chamber 140 is sealed by the outlet valve 141. The outlet valve 141 and the inlet valve 131 are implemented in a technically similar or substantially identical manner. The outlet valve 141 comprises a tubular section 144 with a hollow interior in permanent fluid contact with the dispensing chamber 140. The tubular section 144 may extend distally from the dispensing chamber 140. The tubular section 144 may be stepped down in radial direction compared to the geometry or diameter of the dispensing chamber 140.

Towards the distal end the tubular section 144, hence the sidewall 145 of the outlet valve 141 is confined in distal direction by a closed end face 147. The sidewall 145 also comprises a through opening 146 or several through openings 146 near the distal end face 147. An outside surface of the sidewall 145 is also tightly engaged with another tubular sheath 148, which is elastically deformable at least in radial direction. As soon as a pressure inside the tubular section 144 is larger than a pressure outside the outlet the fluid provided in the dispensing chamber 140 will be urged through the through opening(s) 146 into a gap or a slit provided between the outside surface of the sidewall 146 and an inside surface of the radially widened tubular sheath 148 of elastic material.

This way, the fluid may flow into and through the atomizer 2 enclosing the distal end of the outlet valve 141. With the presently illustrated example the atomizer 2 is rigidly fastened, e.g. snap fitted on the distal end of the nozzle 14 and comprises the outlet orifice 3 located downstream and hence distally from the tubular section 144 of the outlet valve 141. Due to an increase of the fluid pressure inside the dispensing chamber 140 fluid is expelled through the hollow tubular section 144 of the outlet valve 141 through the at least one through opening 146, thereby radially widening the tubular sheath 148 so as to enter the orifice 3 by way of which the fluid expelled through the atomizer 2 is effectively atomized or nebulized.

With other examples (not illustrated) the outlet orifice 3 is in fluid connection with the dispensing chamber 140 and/or with the outlet valve 144 and is void of an atomizer 2. Here, the fluid dispensing device 1 may be configured to dispense other types of fluids, e.g. highly viscous fluids, such as syrups, that do not require to be atomized or nebulized. The outlet orifice 3 may be also configured to produce single or multiple drops or droplets of the fluid in a dispensing action. Generally, the outlet orifice 3 may be arranged the housing 10 or may be integrated into the housing 10 without a nozzle 14.

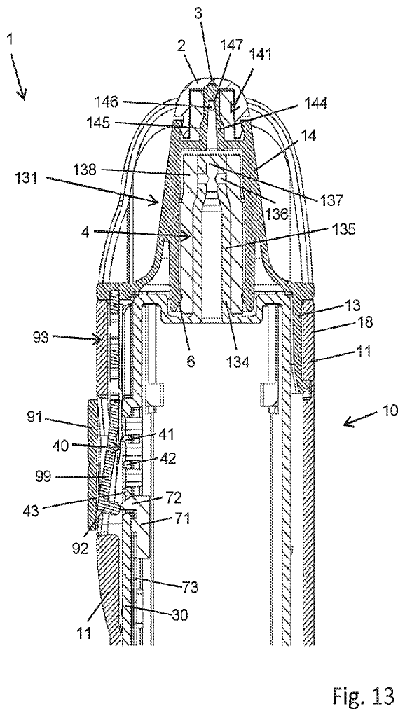

The dispensing chamber 140 can be filled with the fluid by moving the inlet valve 131 in proximal direction relative to the nozzle 14, which is downwardly in the illustration of FIGS. 13-17. In this way and since the outlet valve 141 prevents ingress of air into the dispensing chamber 140 the pressure inside the dispensing chamber drops below the fluid pressure provided inside the fluid container 110, which is in permanent flow connection with the hollow interior of the inner tubular section 134.

Accordingly, and due to the pressure gradient, the fluid will then start to flow through the at least one or several through openings 136, thereby slightly radially outwardly widening the tubular sheath 138. This way, the dispensing chamber 140 will the filled with the fluid.

For dispensing a dose of the fluid by the outlet valve 141 it is intended to longitudinally displace the inlet valve 131 in distal direction towards the outlet valve 141. This way, the volume of the dispensing chamber 140 is reduced and the fluid pressure inside the dispensing chamber 140 will raise. When the inside pressure of the dispensing chamber 140 is larger than an inherent resistance of the outlet valve 141 the rising fluid pressure will be effective to urge the fluid through the through opening(s) 146, thereby radially widening the tubular sheath 148 and expelling the fluid through the atomizer 2. In the sequence of FIGS. 14-17, the temporal order of individual steps during one or repeated dispensing action(s) is schematically illustrated.

In FIG. 14 the dispensing chamber 140 is in an initial configuration, wherein the inlet valve 131 and hence the discharge mechanism 130 is in a biased or initial configuration. The interaction of the discharge mechanism 130, the mechanical energy storage 50, the interlock 70 and the trigger mechanism 90 is implemented such, that numerous discrete doses of the fluid can be dispensed stepwise. After actuating 18 the trigger mechanism 90 for a first time, the driver 30 rigidly connected with the inlet valve 131 is moved in distal direction as illustrated in FIG. 15. Compared to the initial configuration of FIG. 14 the dispensing chamber 140′ comprises a slightly reduced volume, which is due to the distally directed sliding movement of the driver 30 and the inlet valve 131 relative to the nozzle 14 and hence relative to the housing 10.

When the trigger mechanism 90 is actuated a second time, the driver 30 and the inlet valve 131 are subject to a further distally directed discrete movement, thus leading to a further reduction of the volume or size of the dispensing chamber 140″ as illustrated in FIG. 16. After a repeated or after another actuation as shown in FIG. 17, hence after a last available actuation of the trigger mechanism 90 the driver 30 and hence the inlet valve 131 reaches a distal end position, wherein the size of the dispensing chamber 140″′ is at a minimum.

Moving of the inlet valve 131 and hence moving of the driver 30 towards a proximal direction is effective and configured to fill the dispensing chamber 140 with the fluid. Here, a respective amount of the fluid is withdrawn from the interior of the fluid container 110 by way of suction. For dispensing multiple doses or strokes the driver 30 and hence the inlet valve 131 is moved in numerous discrete steps in longitudinal distal direction relative to the outlet valve 141 as illustrated by the sequence of FIGS. 14-17. Here, the fluid located inside the dispensing chamber 140 is expelled through the outlet valve 141 and is atomized by the orifice or 3 of the atomizer 2.

The driver 30 is slidably displaced with regard to the longitudinal direction inside the body 11.

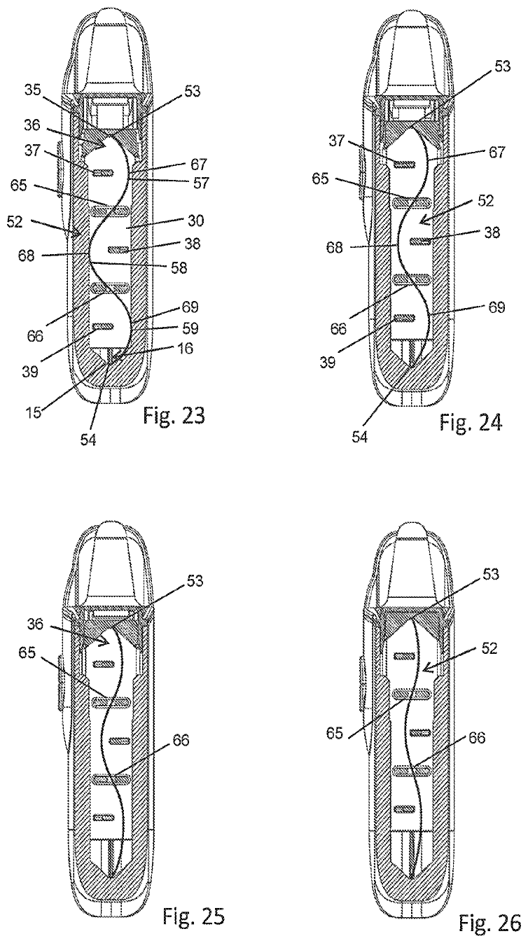

The driver 30 is movable in longitudinal direction under the effect of the mechanical energy storage 50. The driver 30 is also operable to bias or to preload the mechanical energy storage 50. The driver 30 is longitudinally slidably guided in the housing 10 between a biased position as illustrated in FIG. 14 and an unbiased position as illustrated in FIG. 17. The biased configuration is also reflected by FIGS. 23 and 24, whereas the unbiased position is effective illustrated in FIG. 26. When in the unbiased configuration the driver 30 is in a distal end position. In the biased position the driver 30 is in a proximal end position.

The driver 30 is displaceable towards the biased position, hence towards the proximal direction against the action of the mechanical energy storage 50. The driver 30 is movable in the opposite direction under the action of the mechanical energy storage. When the mechanical energy storage 50 releases mechanical energy this mechanical energy is operable to urge or to move the driver 30 in distal direction so as to effectuate a spray discharging by moving the inlet valve 131 relative to the outlet valve 141 as described above.

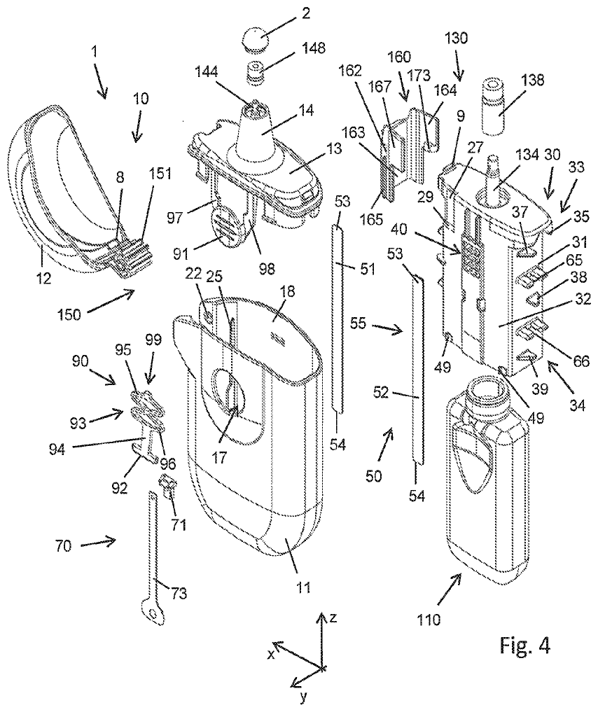

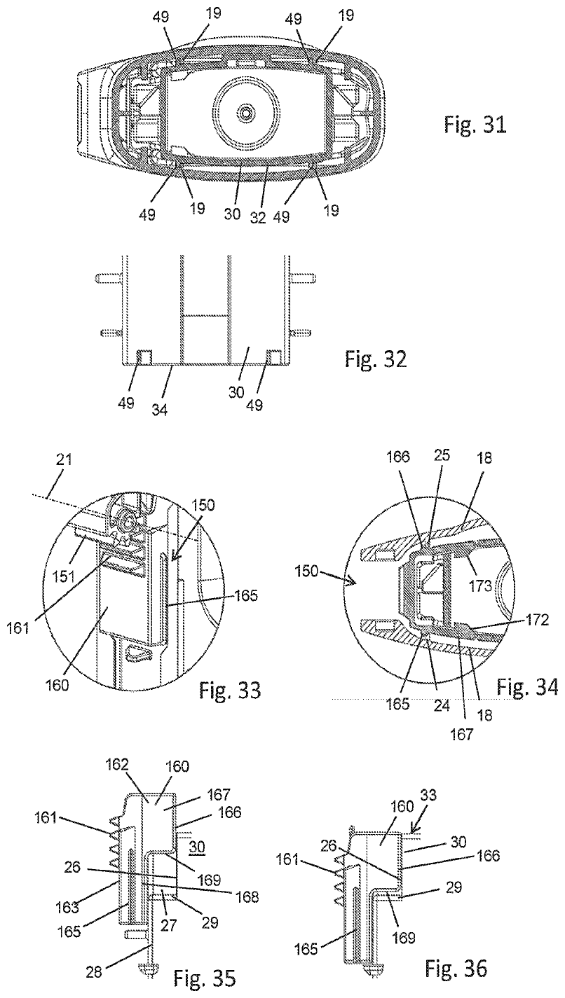

The driver as illustrated in FIG. 4 comprises or forms a kind of an inner housing completely enclosing the fluid container 110. The driver 30 forms a kind of a carrier 31 for the fluid container 110. The fluid container 110 is rigidly fastened or fixed to the driver 30. Since the driver 30 is movably disposed inside the housing 10 it serves as a kind of a movable carrier 31 for the fluid container 110. The driver 30 is longitudinally guided by a sliding engagement with the body 11. As illustrated in greater detail by FIGS. 31 and 32, the sidewall 32 of the driver 30 comprises numerous outwardly protruding guiding protrusions 49. These protrusions 49 may be provided at or near a proximal end 34 of the driver 30. The guiding protrusions 49 are in sliding engagement with complementary shaped longitudinal extending guiding ribs 19 protruding inwardly from the sidewall 18 of the body 11.

With the presently illustrated examples there are provided four outwardly extending guiding protrusions 49 on the outside surface of the sidewall 18 of the driver 30. This way, there can be provided a rather tilt-free and/or cant-free and hence rather smooth longitudinal guiding of the driver 30 inside the body 11 of the housing 10. The driver 30 is movably and slidably displaceable between the unbiased position as illustrated in FIG. 26 and the biased position as illustrated in FIG. 23 or 24. The driver 30 is slidably displaceable relative to the housing 30 and is further in mechanical engagement with the mechanical energy storage 50.

The mechanical energy storage 50 comprises a first drive spring 51 and a second drive spring 52. The first drive spring 51 and the second drive spring 52 are provided on opposite side edges of the driver 30. The driver 30 comprises a continues cross sectional profile extending in the longitudinal direction (z). The driver 30 and hence the carrier 31 comprises a sidewall 32 extending in longitudinal direction and comprises a somewhat rectangular shaped cross-section. A long side of the sidewall extends along a second transverse direction (x) and a short side of the sidewall extends along a first transverse direction (y).

The first and the second drive springs 51 are provided on the opposite side of the sidewall 32 of the driver 30 that are separated along the second direction (x). Towards or near the distal end 33 the driver 30 comprises an abutment 35 with a V-shaped recess 36. A distal end of the recess 36 forms a proximally facing abutment 35 for a respective distally located longitudinal end 53 of the drive spring 51, 52. In the opposite direction and hence towards the distal end the drive springs 51, 52 each comprise a proximal longitudinal end 54 that is in abutment with a distally facing abutment 15 provided at a respective V-shaped recess 16 on the inside surface of the body 11 as indicated in FIG. 21-26. It is self-explaining, that opposite short sides of the sidewall 32 of the driver 30 comprises a somewhat identical geometry with regards to an abutment or engagement with the drive springs 51, 52.

Hence, the driver 30 comprises a driver abutment 35 to engage with the first longitudinal end 53 of the first drive spring 51 and/or of the second drive spring 52. The housing 10 comprises a housing abutment 15 to engage with the second longitudinal end 34 of the drive spring(s) 51, 52.

The mechanical energy storage 50 is reversibly transferable into a preloaded state by resiliently compressing the drive spring(s) 51, 52 in the longitudinal direction. As illustrated in FIGS. 23-26 the drive spring(s) 51, 52 are longitudinally compressible, thereby inducing a resilient deformation of the drive spring 51, 52 along the first transverse direction (y). Each of the drive springs 51, 52 comprises a rather planar shaped longitudinally extending slab profile. The drive springs 51, 52 are deformable into an undulated structure with at least one arc-shaped undulation 57, 58, 59 as indicated in FIG. 21.

This way, the drive spring 51, 52 are compressible into a S-shaped, double S-shaped or M-shaped deformed configuration. In order to induce a well-defined transverse deformation of the drive springs 51, 52 at least one of the driver 30 and the housing 10 comprises a spring fixing notch 65, 66 through which the longitudinal extending slot profile of the drive spring 51, 52 is guided and/or fixed in longitudinal direction (z).

A free space of the spring fixing notch is 65, 66, through which the drive spring 51, 52 is longitudinally guided is only slightly larger than a thickness of the lateral profile of the drive spring 51, 56. Hence, in the region of the spring fixing notches 65, 66 the position of the drive spring 51, 52 is substantially fixed with regards to the first transverse direction (y).

The spring fixing notches 65, 66 are separated in longitudinal direction. This way, and when the oppositely located longitudinal ends 53, 54 of the drive spring(s) 51, 52 are subject to a compression in longitudinal direction (z) there will evolve oppositely directed arc-shaped undulations 57, 58, 59 extending in the first transverse direction (y). The undulations are provided by respective deformable portions 67, 68, 69 of the respective drive springs 51, 52.

As illustrated in FIGS. 21-26 a first deformable or bendable portion 67 of the drive spring 52 is provided between the driver abutment 35 and the first spring fixing notch 65. The second spring fixing notch 66 is provided at a longitudinal distance in proximal direction from the first spring fixing notch 65. Between the first spring fixing notch 65 and the second spring fixing notch 66 there extends a second bendable portion 68 of the drive spring 52, which forms a second arc-shaped undulation 58. The second undulation 58 extends in the first transverse direction (y) opposite to the extension of the first undulation 57 as provided by the first deformable or bendable portion 67 of the drive spring 52.

Between the second spring fixing notch 66 and the housing abutment 15 there is located a third bendable or deformable portion 69 of the drive spring 52. When subject to longitudinal compression the third bendable portion 69 also forms an arc-shaped undulation 59 extending in the same direction as the first undulation 57.

On the outside surface of the sidewall 32 of the driver 30 there are further provided spring deformation guiding elements 37, 38 and 39 that are located e.g. midway between adjacently arranged prefixing notches 65, 66 and between an upper or lower prefixing large and a respective abutment 15, 35 of the housing 10 and/or of the driver 30. A first spring deformation guiding element 37 is located longitudinally between the driver abutment 35 and the first prefixing notch 65. A second spring deformation guiding element 38 is located longitudinally between the first spring fixing notch 65 and the second spring fixing notch 66 and a third spring deformation guiding element 39 is located, e.g. longitudinally midway, between the second spring fixing notch 66 and the housing abutment 15.

Spring deformation guiding elements positioned adjacently in longitudinal direction (z) are located on opposite sides of the drive spring 51, 52 as seen with regards to the first transverse direction (y). The spring deformation guiding elements 37, 38, 39 are configured to induce a deformation of the first, second and third deformable or bendable portions 67, 68, 69 of the drive spring 51, 52 away from the respective spring deformation guiding element 37, 38, 39 into a respective arc-shaped undulation 57, 58, 59.

Insofar, the spring deformation guiding elements 37, 38, 39 are arranged and configured to break the longitudinal symmetry of the rather straight shaped elongated first and second drive springs 51, 52. A side edge of the spring deformation guiding elements, which protrude from the sidewall 32 of the driver 30 with regard to the second transverse direction (x) are arranged slightly offset from a virtual longitudinal center line of the first and second drive springs 51, 52 as seen in the first transverse direction (y).

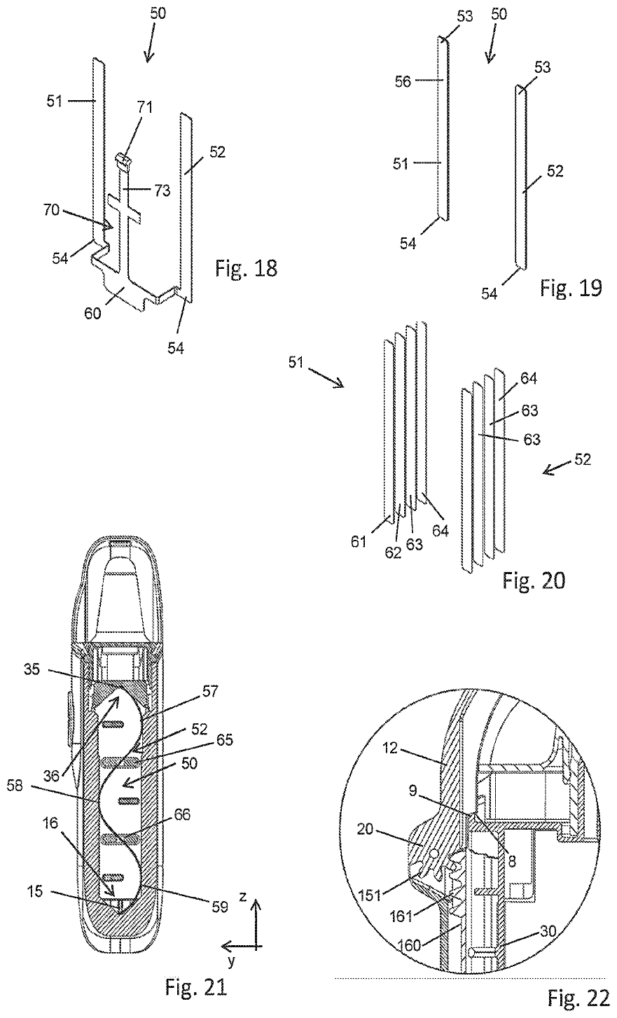

This way, the drive springs 51, 52, which may be of substantially straight shape when in the completely unbiased position as illustrated in FIGS. 18 and 19 are likely to become deformed or slightly prestressed as they are installed or arranged inside the fluid dispensing device 1.

By way of the V-shaped recesses 16, 36 as provided by the housing 10 and the driver 30, a rather precise abutment and alignment of the drive springs 51, 52 can be provided with regards to the first transverse direction (y). The V-shaped recesses 16, 36 provide a kind of a self-centered arrangement of the drive Springs 51, 52 with regard to the first transverse direction (y).

The drive springs 51, 52 as illustrated in FIGS. 19 and 20 may comprise a stamped or punched sheet metal. With some examples and as illustrated in FIG. 20, the drive spring 51 may comprise numerous spring elements, such as a first spring element 61, a second spring element 62 and further spring elements 63, 64 that are mutually fixed, bonded, welded, fused, or laminated to form or constitute the drive spring. Such multiple springs allow to design and to obtain optimal force profiles and to increase the resistance to material yield.

In effect, the longitudinally extending elongated and rather straight shaped drive springs 51, 52 are beneficial to provide a rather constant spring force in longitudinal direction (z) when subject to the deformation with regards to the first lateral direction (y). Rather independently of the degree of deformation in the first lateral direction (y) as illustrated in the various configurations of FIGS. 23-26 the force effect and the force provided in longitudinal direction (z) between the oppositely located longitudinal ends 53, 54 is substantially constant. This is of particular benefit to provide a rather constant driving force for moving the driver 30 relative to the housing 10.

The spring arrangement is further of particular benefit to provide a sequence of dispensing actions without an intermediate charging or reloading of the mechanical energy storage 50. Hence, the mechanical energy stored by the drive springs 51, 52 and hence stored by the mechanical energy storage 50 can be released in a sequence of discrete steps, each of which releasing an amount of mechanical energy sufficient to effectuate a spray discharging of a dose of the fluid.

Apart from that, the longitudinal and rather elongated straight shape of the drive springs 51, 52 is beneficial with regards to a compact design of the mechanical energy storage 50. The drive springs 51, 52 only require a rather limited construction space.

In the example of FIG. 19 the mechanical energy storage 50 comprises two individual drive springs 51, 52, that are separately arranged inside the housing 10. With the further example of FIG. 18 the drive springs 51, 52 are mutually connected by a crossbar 60 extending along the second transverse direction (x). By way of the crossbar 60, the first and the second drive springs 51, 52 become part of a spring assembly. They may be integrally formed. The entire drive spring assembly as illustrated in FIG. 18 may be integrally formed from a single sheet metal. The drive spring assembly may also comprise numerous spring elements 61, 62, 63, 64. Here, a laminated sheet metal may be punched and/or stamped and/or embossed accordingly in order to provide or to form the rather specific geometric structure of the crossbar 60. As illustrated, the crossbar 60 interconnects the longitudinal ends, e.g. the proximal longitudinal ends 54 of the drive springs 51, 52.

With the integrated drive spring assembly is also conceivable to implement a further spring element 73 into the drive spring assembly. The further spring 73 may belong to the releasable interlock 70 and may serve to keep a locking element 71, e.g. provided as a free end of the slab-like locking spring 73 in engagement with a complementary shaped counter locking structure 40 of the driver 30 as will be explained further below.

Here, all metal components of the fluid dispensing device 1 may be integrated in the drive spring assembly, thereby facilitating the mass manufacturing and assembly of individual parts of the fluid dispensing device 1. Also, the number of individual parts for assembly of the device 1 can be reduced.

As will be explained and described further below the locking element 71 of the interlock 70 is operable to retain the mechanical energy stored in the mechanical energy storage 50. The releasable interlock 70 is operably engaged with the trigger mechanism 90. Actuation of a trigger button 91 may at least temporally disengage the locking element 70 from the counter locking structure 40 and may thus allow to release at least a portion of the mechanical energy from the mechanical energy storage 50 in order to move the driver 30 towards the unbiased position, thereby dispensing a dose of the fluid.

In the illustration of FIG. 21 the protective cap 12 is in the closed position. Here and as shown in greater detail in FIG. 22 an abutment 8 provided on an inside surface of the cap 12, e.g. located in close vicinity to the hinge 20, directly engages with a complementary-shaped counter abutment 9 as provided on a distal end of the driver 30. The counter abutment 9 may comprise an upwardly or distally extending protrusion.

When the protective cap 12 is about to reach the closed position the abutment 8 gets in direct mechanical contact with the counter abutment 9. When reaching the closed position the abutment 8 is effective to press down onto the counter abutment 9 and to exert a respective proximally directed force effect onto the counter abutment 9, thereby inducing a further proximally directed movement of the driver 30 towards the proximal direction.

This leads to a kind of an over-pressing of the mechanical energy storage 50. As illustrated with this kind of a primed configuration as shown in FIG. 21 the undulations 57, 58, 59 may reach or get into abutment with oppositely located inside surface sections of the body 11. This over-pressing function further serves to move the driver 30 even is further into the proximal direction, thereby unloading the engagement of the locking element 71 and hence of the releasable interlock 70 with the driver 30 or counter locking structure 40. By opening of the protective cap 12 as illustrated in FIG. 23 the abutment 8 and the counter abutment 9 get out of engagement and the driver 30 is moved slightly in distal longitudinal direction until the interlock 70 gets in engagement with the driver 30 and hence until the locking element 71 gets into abutment or engagement with the counter locking structure 40.

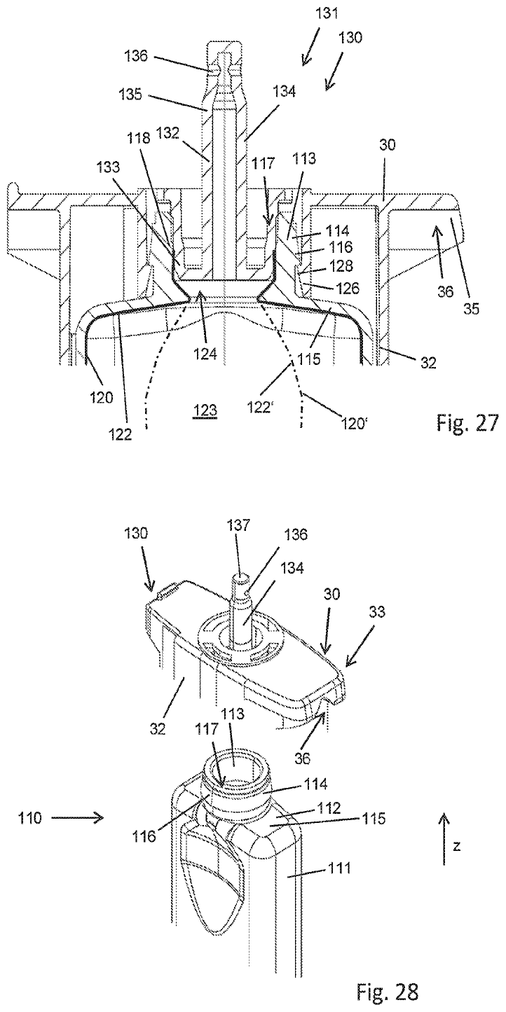

In FIGS. 27-30 there are illustrated two examples of a fluid container 110 to be used with the fluid dispensing device 1. The fluid container 110 comprises a flexible bag 120 with a flexible sidewall 122. The flexible bag 120 comprises or forms an interior volume 123 to be filled with the fluid. The flexible bag 120 further comprises a bag outlet 124 towards a distal end. The bag outlet 124 may be formed by a longitudinal end of the flexible sidewall 122. The fluid container 110 further comprises a rigid fastening adapter 112 that comprises a fastening structure 114 for mechanical engagement with a corresponding or complementary-shaped counter fastening structure 126 of the fluid dispensing device. Typically, the counter fastening structure 126 is provided by or integrated into the driver 30. The rigid fastening adapter 112 further comprises an outlet shaft 113 in fluid communication with the interior volume 123 confined by the flexible bag 120. Typically, the outlet shaft 113 is a hollow shaft configured to guide the fluid there through.

When the fastening structure 114 of the rigid fastening adapter 112 engages with the complementary or correspondingly shaped counter fastening structure 126 of the fluid dispensing device 1 there is provided a fluid-tight connection between the fluid discharge mechanism 130 of the dispensing device 1 and the outlet shaft 113 as provided by the rigid fastening adapter 112.

The flexible bag 120 provides a rather easy and smooth withdrawal of the fluid from the interior volume 123. When withdrawing a portion of the fluid from the interior volume 123, the flexible bag 120 may collapse due to the reduced interior volume 123. As indicated in FIG. 27, and when more and more fluid is withdrawn from the interior volume 123, the sidewall 122′ and the flexible bag 120′ change their shape towards a collapsed configuration.

A collapsible sidewall 122 of the flexible bag 120 and hence a collapsible fluid-tight bag 120 allows and supports a suction-based withdrawal of the fluid from the interior volume 123.

The rigid fastening adapter 112 that is sealingly engaged with the bag outlet 124 provides a well-defined mechanical fastening of the fluid container 110 with the discharge mechanism 130.

As indicated in FIGS. 27-29 the fastening structure 114 of the fluid container 110 is provided on an outside facing portion of the outlet shaft 113 of the rigid fastening adapter 112. The fastening structure 114 may comprise one or several snap elements 116 configured to mechanically engage with a complementary-shaped or with numerous complementary-shaped counter snap elements 128 as provided on the driver 30.

With the example of FIGS. 27 and 28 an outside surface of the rather tubular shaped hollow outlet shaft 113 is provided with fastening structure 114 implemented as a snap element 116 comprising a beveled side edge or side flank terminating in proximal direction into a stepped abutment face to engage with a complementary shaped stepped counter abutment face of the counter fastening structure 126. This way, a kind of a snap fit connection can be provided between the rigid fastening adapter 112 and the driver 30 and hence with the fluid discharge mechanism 130.

The fastening structure 114 and hence the beveled shaped snap element 116 may comprise an annular structure to engage with a complementary shaped annular structure of the counter snap element 128. In order to enable a rather smooth and easy mutual snap-fit engagement there may be provided at least one or several interruptions or recesses in the annular structure of at least one of the beveled shaped snap element 116 and the complementary shaped beveled counter snap element 128. As illustrated in the cross-section of FIG. 27, the snap element 116 comprises a barb-shaped structure and the complementary shaped counter snap element 128 comprises a corresponding barb-shaped structure. This way, the mutual engagement of the fluid container 110 with the discharge mechanism 130 can be easily provided simply by pushing the fluid container 110 with its hollow outlet shaft 113 in distal direction into or against the driver 30 of the discharge mechanism 130.

As particularly illustrated in FIGS. 27 and 28 the driver 30 comprises a tubular shaped valve insert 132 configured for insertion into the hollow outlet shaft 113 of the rigid fastening adapter 112. The valve insert 132 comprises the above-mentioned inner tubular section 134 of the inlet valve 131. In other words, the inlet valve 131 may be integrated into the driver 30. The valve insert 132 comprises an outer sleeve section 133 complementary shaped to an inside surface of the hollow outlet shaft 113. The outlet shaft 113 comprises a tubular shaped receptacle 117 towards the distal direction so as to receive the valve insert 132. The receptacle 117, in particular an inside facing sidewall section of the receptacle 117, comprises a tapered or conically shaped seal seat section 118 to engage with a complementary shaped tapered counter seal seating section of the valve insert 132.

The outside surface of the tubular shaped valve insert 132 and the inside surface of the receptacle 117 of the outlet shaft 113 are configured such that a fluid tight engagement is provided between the bag outlet 124 and the hollow portion of the valve insert 132 as the fastening structure 114 engages with the complementary-shaped counter fastening structure 126.

With another example occurs in not illustrated it is also conceivable, that the inlet valve 131 comprises a hollow shaft with a receptacle configured for receiving and insertion of the outlet shaft 113 of the fluid container 110.

As further illustrated in FIG. 27 the rigid fastening adapter 112 comprises a shoulder portion 115 adjacent to the distal end of the flexible bag 120. The shoulder portion 115 merges into the distally extending outlet shaft 113. The flexible bag 120 conforms and adapts to the shape of the shoulder portion 115 and the outlet shaft 113. An open end of the sidewall 122 of the flexible bag 120 is located in the interior of the outlet shaft 113. In particular, the distal end of the sidewall 122 of the flexible bag 120 ends at the outlet 124 and is sealingly engaged with the inside surface of the hollow outlet shaft 113. It with some examples, an outside surface of the bag outlet 124 and/or an outside surface of the flexible sidewall 122 may be sealed or welded with an inside surface of the cylindrical receptacle 117 of the outlet shaft 113. The bag outlet 124 may be located in or on the tapered seal seat section 118 of the outlet shaft 113. This way, there can be provided a direct fluid tight engagement between the valve insert 132 of the fluid discharge mechanism 130 and the flexible bag 120.

With the example of FIGS. 27 and 20 a rigid fastening adapter 112 forms or comprises an outer rigid casing 111 sized to accommodate the entirety of the flexible bag 120. This way, the rigid casing 111 provides an improved mechanical and/or chemical or physical protection for the flexible bag 120. This may be of particular benefit for manufacturing, transportation, and storage. With some examples the fluid container 110 is releasably connectable to the fluid discharge mechanism 130. Here, the fluid dispensing device 1 may be implemented as a reusable device, wherein an empty fluid container 110 can be replaced by a new one. With other examples the fluid dispensing device 1 is implemented as a disposable device. Here, and when the rigid container 110 is empty the entire fluid dispensing device 1 may be intended to be discarded in its entirety.

With some examples the outer rigid casing 111 is made of a material or a material composition comprising at least one of a high-density polyethylene and a polypropylene. With some examples the outer rigid casing comprises a multilayer structure with a first layer made of a high-density polyethylene and a second layer made of a polypropylene. The lexical bag 120 may be blow molded or injection molded into the outer rigid casing 111. With other examples the flexible bag 120 and the outer rigid casing 111 co-extruded. Any of these manufacturing methods may have certain advantages for a cost efficient and reliable mass manufacturing of such fluid containers.

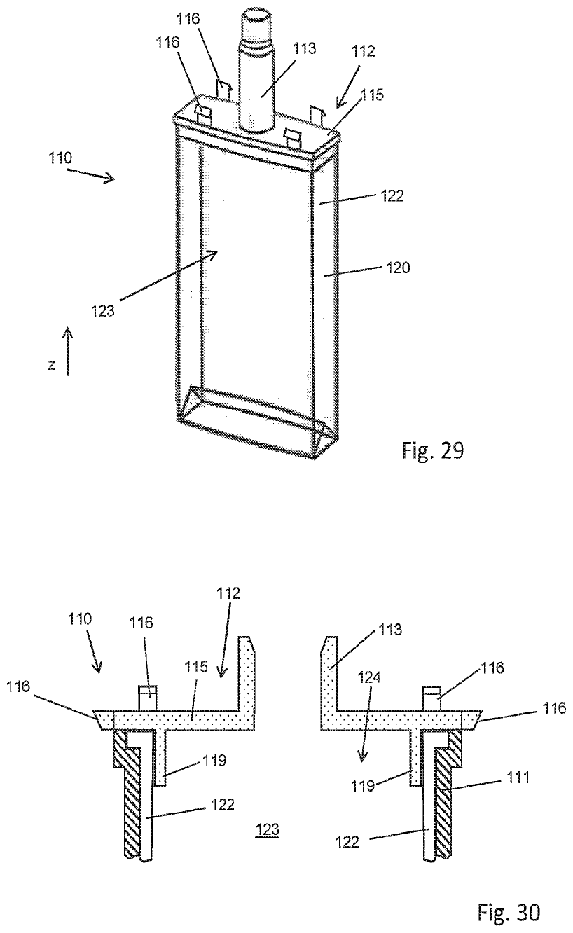

With the example of FIGS. 29 and 30 the fluid container 110 only optionally comprises an outer rigid casing 111. Here, the flexible bag 120 comprises a somewhat rectangular or oval cross section and a continuous sidewall profile. A distal end of the sidewall 122 may be sealingly connected with the rigid fastening adapter 112. Here, the rigid fastening adapter 112 comprises a somewhat planar-shaped board or plate forming the above-mentioned shoulder portion 115.

The rigid fastening adapter 112, comprises the hollow outlet shaft 113 protruding outwardly from the interior volume 123 of the flexible bag 120. An inside facing side of the shoulder portion 115 is in a sealing engagement with the flexible sidewall 122. Here, the shoulder portion 115 comprises numerous snap elements 116 protruding outwardly in distal direction from the shoulder portion 115. Alternatively or additionally, there may be provided respective snap elements 116 at a lateral side edge of the shoulder portion 115. Towards the inside the shoulder portion 150 may comprise a comparatively short sidewall portion 119 extending in longitudinal direction, e.g. forming a circumferentially closed rim.

The sidewall portion 119 may be in abutment with the longitudinal, hence with the distal end of the sidewall 122 of the flexible bag 120. Here, an inside surface of the sidewall 122 may be sealingly engaged with an outside surface of the sidewall portion 119. Alternatively, an outside surface of the sidewall is sealingly engaged with an inside surface of the sidewall portion 119.

Optionally, the fastening adapter 112 and hence the rather planar-shaped shoulder portion 115 may be connected with a cup-shaped rigid casing 111 as illustrated in the cross-section of FIG. 30. The rigid casing 111 may be provided separately and may be mechanically fixed to the fastening adapter 112. Here, the rigid fastening adapter 112 may be provided as a first component, the flexible bag 120 may be provided as a second component and the outer rigid casing 111 may be provided as a third component. For producing and manufacturing the fluid container 110, the three components are mutually assembled and mutually sealed, e.g. welded or otherwise bonded to each other.

Even though not particularly shown, also here the sidewall 120 may comprise a bag outlet 124 comprising a diameter that is smaller than the diameter of the sidewall 122 near a proximal end or in a longitudinal middle portion of the sidewall 122. Also here, and as illustrated in the example of FIG. 27 the bag outlet 124 may be separately sealed and attached to an inside surface of the hollow outlet shaft 113.

Charging and/or preloading of the mechanical energy storage 50 is described below in further detail. For biasing or charging of the mechanical energy storage 50, there is provided a biasing mechanism 150 comprising a biasing member 160 as shown in FIG. 4. The biasing member 160 is operationally coupled to the protective cap 12 and is selectively engageable with the mechanical energy storage 50 to transfer the mechanical energy storage 50 into the preloaded state when the protective cap 12 moves into the closed position.

The biasing mechanism 150 with the biasing member 160 comprises a pinion segment 151 connection to or integrated into the protective cap 12. The biasing member 160 further comprises a rack segment 161 with numerous teeth engaged with the pinion segment 151, e.g. engaged with the teeth of the pinion segment. As illustrated in greater detail in FIGS. 33-39 the individual teeth of the pinion segment 151 mate with complementary shaped teeth of the rack segment 161 of the biasing member 160. The protective cap 12 is connected to the housing 10 by a hinge 20 and is pivotable relative to the housing 10 with regards to a hinge axis 21, wherein a radial center of the curved pinion segment 151 substantially coincides with the hinge axis 21.

The rack segment 161 comprises numerous teeth that are arranged next to each other along the longitudinal direction (z). The rack segment 161 is of rather elongated shape and extends along the longitudinal direction. As the protective cap 12 is subject to a pivoting motion relative to the housing 10 the teeth of the pinion segment 151 successively engage with the teeth of the rack segment 161, thereby inducing a longitudinal sliding motion of the rack segment 161 and hence of the biasing member 160 relative to the housing 10 and relative to the body 11. The biasing member 160 comprises a somewhat U-shaped profile as seen in the transverse cross-section.

The biasing member 160 comprises a first sidewall section 162, a second side wall section 163 and a third sidewall section 164, wherein the first and the third sidewall sections 162, 164 extend substantially parallel to each other. They are separated with regards to the first transverse direction (y). The second sidewall section 163 extends between the first and the third side wall sections 162, 164. The numerous sidewall sections 162, 163, 164 are integrally formed. Hence, the biasing member 160 is implemented as a single piece.

On the outside surfaces of the first and the third sidewall sections 162, 164 there are provided longitudinal extending guiding ribs 165, 166 to engage with complementary shaped guiding ribs 24, 25 as provided on an inside surface of the sidewall 18 of the body 11. This way, the biasing member 160 is longitudinally guided in the body 11 of the housing 10. By way of a pair wise mutual engagement of guiding ribs 166, 25 and guiding ribs 165, 24 a rather smooth, tilt-free and/or cant-free longitudinal sliding displacement of the biasing member 160 relative to the body 11 can be provided.

The first sidewall section 162 further comprises a lateral protrusion 167 extending and protruding along the second lateral direction (x) from a lower portion of the second sidewall 162. The lateral protrusion 167 lies in the plane of the second sidewall section and forms an abutment face 169 facing in proximal direction. The abutment face 169 is formed by a lower edge of the lateral protrusion 167 and is complementary shaped to a counter stop face or counter abutment face 29 of the driver 30. Here, the driver 30 comprises a longitudinally recess 27 adjoining a distal end 33 of the side wall of the driver 30. The longitudinally extending recess 27 is provided in an outside section of the sidewall 32. It is complementary shaped to the lateral protrusion 167 and provides a supplemental guiding function for the longitudinal sliding motion of the biasing member 160.

As it is further apparent from FIGS. 4 and 34 the third sidewall section 164 is complementary shaped to the first sidewall section 162. It also comprises a respective lateral protrusion 167 with a proximally facing edge forming a respective abutment face to engage with a complementary shaped abutment face of a respective longitudinally extending recess provided on the opposite side wall 32 of the driver (not illustrated). The proximally facing edges of the lateral protrusions one 167 each comprise an inwardly protruding projection 172, 173 by way of which an improved longitudinal abutment can be provided with the driver 30.

During a closing motion of the protective cap 12 and when the driver 30 is in the upper or distal end position, which coincides with the unbiased position of the driver 30, the pivoting motion of the protective cap 12 towards the closed position leads to a respective rotation of the pinion segment 151 which is directly transferred into a longitudinal sliding displacement of the biasing member 160 in longitudinal proximal direction relative to the body 11. In this configuration the proximally facing abutment or side edge 169 is in longitudinal abutment with a complementary shaped counter stop face 29 of the driver 30.

As the protective cap 12 is moved further towards the closed position the biasing member 160 applies a respective proximally directed force effect onto the driver 30, thereby moving the driver 30 against the action of the mechanical energy storage 50 into the proximal end position, hence into the biased position. When reaching the biased position the driver 30 engages with the interlock 70 by way of which the driver 30 is prevented from moving towards the distal direction, hence into the unbiased position. A re-opening of the protective cap 12 may then be accompanied by a respective distally directed motion of the biasing member 160 as illustrated in FIG. 35. Accordingly, the side edge 169 separates from the counter stop face 29 and the driver 30 is free to move in numerous discrete steps in distal direction until a repeated abutment configuration as illustrated in FIG. 36 is reached again.

From FIG. 35 it is further apparent, that the driver comprises an outer side edge 28 that is in sliding engagement with a lower part of the first and third sidewall sections 162, 164 of the biasing member 160. Moreover, also the lateral protrusion 167 of the first and second sidewall sections 162, 164 comprise a respective side edge 166 that is and remains in sliding engagement with a complementary shaped side edge 26 of the recess 27. This way, the side edge 168 of the lower portion of the first and the sidewall sections 162, 164 is and remains in sliding engagement with the lateral side edge 28 of the sidewall 32 of the driver 30 and the lateral side edge 166 of the lateral protrusion 167 of the first and the second sidewall sections 162, 164 is and remains in sliding engagement with a longitudinally extending side edge 26 of the recessed portion 27 of the sidewall 32 of the driver 30. This way, there can be provided an improved tilt-free and/or cant-free sliding displacement of the biasing member 160.

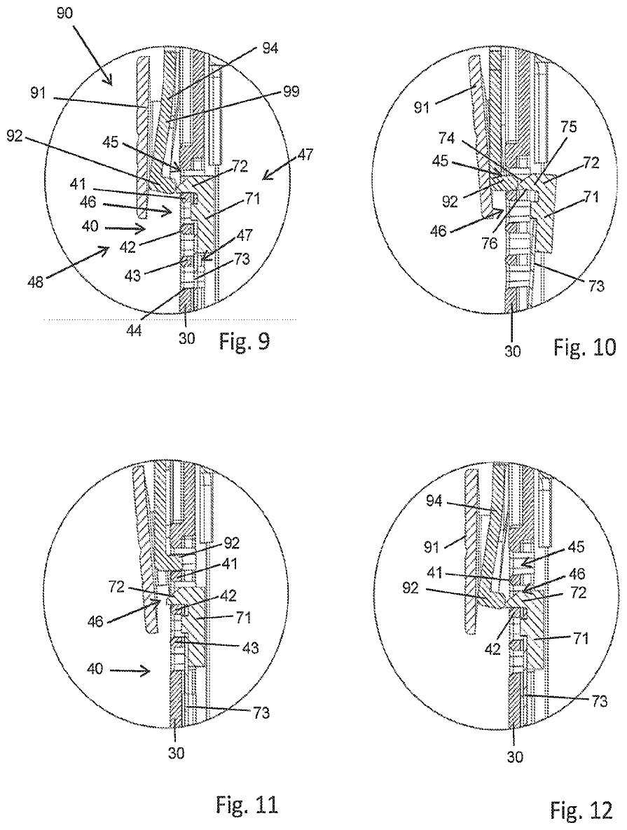

In the following, interaction between the trigger mechanism 90 and the releasable interlock 70 for producing a sequence of dose dispensing procedures is described in greater detail. The driver 30 comprises a counter locking structure 40 on the sidewall 32. The counter locking structure 40 comprises numerous counter locking elements 41, 42, 43, 44 that are separated along the longitudinal direction (z). The interlock 70 comprises a locking element 71 sized and configured to engage with each one of the counter locking elements 41, 42, 43, 44. The mutual interaction between the locking element 71 with each one or with several of the counter locking elements 41, 42, 43, 44 is apparent by the sequence of FIGS. 9-12.

The locking element 71 of the releasable interlock 70 is provided on a longitudinal end of an elongated locking spring 73. The locking spring 73 serves to urge or to keep the locking element 71 in engagement with a counter locking structure 40. In the present case the locking spring 73 serves to displace the locking element 71 in the first transverse direction (y). The locking element comprises a pawl 72 configured to engage into recesses 45, 46 provided longitudinally between the row or sequence of the counter locking elements 41, 42, 43, 44. Towards the distal direction the free end of the locking element 71 comprises a beveled edge 74. This way and as the driver 30 provided with the counter locking structure 40 is subject to a longitudinal sliding displacement towards the proximal direction the beveled edge 74 slides along the sequence of counter locking elements 44, 43, 42, 41 and is thereby deflected against the action of the locking spring 73.

When the driver 30 has reached the biased position, and hence when the driver 30 is in a proximal end position the locking element 71 is in engagement with a first counter locking element 41. Here and as illustrated in FIG. 9 the protruding portion of the locking element 71 is located inside a first recess 45 and effectively blocks and prevents a distally directed movement of the driver 30.

As indicated in FIGS. 9-12 the interlock 70 and the locking element 71 are located on a first side 47 of the through recess 45. On an opposite second side 48 of the through recess 45 there is aligned a trigger head 92 of a trigger member 99. The trigger member 99 and in particular the trigger head 92 protruding from the trigger member 99 is longitudinally aligned with the retaining pawl 72 of the locking element. The trigger button 91 is also in transverse engagement with the trigger member 99, in particular with the trigger head 92.

Depression of the trigger button 91 leads to an insertion of the trigger head 92 into the second side 48 of the through recess 45, thereby urging the retaining pawl 72 out of the respective recess 45 as illustrated in FIG. 10. In this configuration the interlock 70 is disengaged from the counter locking structure 40 of the driver 30 and the driver 30 is hence free to move in distal direction under the action of the relaxing drive springs 51, 52. Since the retaining pawl 72 is biased outwardly, hence towards the first transverse direction (y) by the locking spring 73 the retaining pawl 72 immediately engages with a n adjacently located second through opening 46 of the counter locking structure 40 as illustrated in FIG. 11.

Here, the retaining pawl 72, e.g. its free end 75, enters the through recess 46 and engages with its stop face 76 with the second counter locking element 42. Consequently, the dispensing motion of the driver 30 towards the unbiased position is stopped. During this distally directed longitudinal sliding movement of the driver 30 the trigger button 91 may still remain in the depressed configuration as illustrated in FIG. 11.

The trigger button 91 is attached to the housing 10. It may be integrally formed with the closure 13. As illustrated in FIG. 4, the trigger button 91 is movable from an idle position as illustrated in FIG. 9 into a trigger position as shown in FIGS. 10 and 11 against the action of a resilient member 97, 98. Here, there are provided two resilient members 97, 98 that resiliently deformable. They provide a fixing and connection of the trigger button 91 to the closure 13. The trigger button 91 extends through an aperture 17 provided in the sidewall 18 of the body 11. The resilient members 97, 98 are located inside the cavity formed by the body 11. Accordingly, the trigger button 91 is depressible inwardly against the return action of the resilient members 97, 98.

The inwardly directed depression of the trigger button 91 urges the trigger head 92 into one of the through recesses 45, 46 as provided by the counter locking structure 40. When the driver 30 is subject to a distally directed dispensing motion while the trigger button 91 one is still depressed the trigger head 92 remains trapped in the respective through recess 45 as illustrated in FIG. 11. The trigger member 99 is deformable in longitudinal direction (z) and is particularly compressible in the longitudinal direction.

As shown in detail in FIG. 4, the trigger member 99 comprises the trigger head 92 that forms a proximal end of the trigger member 99. Towards the upper or opposite end of the trigger member 99 there is provided a trigger spring 93, e.g. with a first and a second spring segment 95, 96 that are compressible in longitudinal direction against the action of a respective return spring force. The trigger spring 93 may be compressed as the trigger is 92 is subject to a distally directed motion while located in a recess 45, 46 or while in engagement with the counter locking structure 40. The trigger spring 93 is connected to the trigger head 92 by a longitudinal extending trigger extension 94. The trigger member 99 may be made of an elastic material. It may comprise a plastic material or a metallic component.

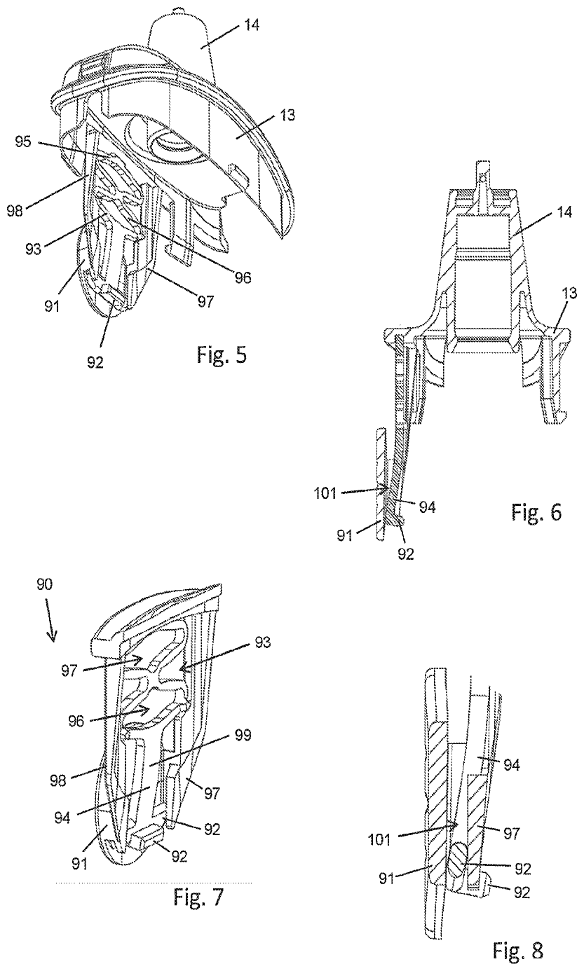

Now and when the trigger button 91 is released the resilient members 97, 98 serve to deflect the trigger button 91 into the initial configuration. As becomes apparent from the illustration of FIGS. 5-8, the trigger head 92 is longitudinally guided in a sliding or guiding groove 101 provided between the resilient members 97, 98 and the inside surface of the trigger button 91. This way, and when the trigger button 91 is returning into the initial position the trigger head 92 moves from the trigger position as illustrated in FIGS. 10 and 11 into its idle position as shown in FIGS. 9 and 12. Reaching the idle position disengages the trigger head 92 from the outer locking structure 40 and allows a relaxing of the trigger spring 93 into an initial position or initial configuration.

This way, the trigger head 92 returns into an initial configuration or initial position relative to the trigger button 91. Since in effect, the relative position of the trigger head 91 to the trigger button 91 is the same in both configurations of FIG. 9 and FIG. 12. The difference in the configurations of FIGS. 9 and 12 is that the driver 30 has moved in distal direction, hence towards the unbiased position by a discrete step, which step size is defined by the distance of longitudinally adjacently located counter locking elements 41, 42, 43, 44 of the counter locking structure 40.

Accordingly, and when the trigger button 91 is released in FIG. 11 the trigger head 92 returns into an initial position due to the relaxation of the trigger spring 93 and properly aligns with the second through recess 46 as provided by the counter locking structure 40. Accordingly, the trigger head 92 is in alignment with the retaining pawl 72 located in the second through recess 46. Now and when the trigger button 91 one is depressed again the trigger head 92 urges the retaining pawl 72 out of engagement with the counter locking structure 40 thereby allowing and supporting a further distally directed dispensing motion of the driver 30 towards the unbiased position.

This way, the trigger mechanism 90 can be actuated at least two times or even several times thereby releasing only a portion of the mechanical energy stored in the mechanical energy storage 50. Between repeated actuations of the trigger mechanism 90 it is not necessary to reload or to recharge the mechanical energy storage 50. Once the user has opened the protective cap 12 the fluid dispensing device 1 can be readily used to dispense a first dose of the fluid e.g. in a first nostril and to subsequently dispense a second dose of the fluid into a second nostril.

While the invention has been described and illustrated herein by references to various specific materials, it is understood that the invention is not restricted to the combinations of material and procedures selected for that purpose. Numerous variations of such details can be implied as will be appreciated by those skilled in the art. It is intended that the specification and examples be considered as exemplary, only, with the true scope and spirit of the invention being indicated by the following claims.

### REFERENCE NUMBERS

1 fluid dispensing delivery device
2 atomizer
3 orifice
4 hollow shaft
5 snap feature
6 sealing rib
7 counter snap feature
8 abutment
9 counter abutment
10 housing
11 body
12 protective cap
13 closure
14 nozzle
15 abutment
16 recess
17 aperture
18 sidewall
19 guiding rib
20 hinge
21 hinge axis
22 recess
24 rib
25 rib
26 side edge
27 recess
28 side edge
29 counter stop face
30 driver
31 carrier
32 sidewall
33 distal end
34 proximal end
35 abutment
36 recess
37 guiding element
38 guiding element
39 guiding element
40 counter locking structure
41 counter locking element
42 counter locking element
43 counter locking element
44 counter locking element
45 recess
46 recess
47 first side
48 second side
49 guiding protrusion
50 mechanical energy storage
51 drive spring
52 drive spring
53 longitudinal end
54 longitudinal end
55 buckling spring
56 spring rod
57 undulation
58 undulation
59 undulation
60 cross bar
61 spring element
62 spring element
63 spring element
64 spring element
65 fixing notch
66 fixing notch
67 deformable portion
68 deformable portion
69 deformable portion
70 interlock
71 locking element
72 pawl
73 locking spring
74 beveled edge
75 free end
76 stop face
90 trigger mechanism
91 trigger button
92 trigger head
93 trigger spring
94 trigger extension
95 spring segment
96 spring segment
97 resilient member
98 resilient member
99 trigger member
101 guiding groove
110 fluid container
111 rigid casing
112 fastening adapter
113 outlet shaft
114 fastening structure
115 shoulder portion
116 snap element
117 receptacle
118 seal seat section
119 sidewall
120 flexible bag
122 sidewall
123 interior volume
124 bag outlet
126 counter fastening structure
128 counter fastening element
130 discharge mechanism
131 inlet valve
132 valve insert
133 outer sleeve section
134 inner tubular section
135 sidewall
136 through opening
137 end face
138 tubular sheath
139 end face
140 dispensing chamber
141 outlet valve
142 sidewall
144 tubular section
145 sidewall
146 through opening
147 end face
148 tubular sheath
150 biasing mechanism
151 pinion segment
152 protrusion
160 biasing member
161 rack segment
162 sidewall section
163 sidewall section
164 sidewall section
165 rib
166 rib
167 protrusion
168 side edge
169 side edge
172 projection
173 projection

1. A fluid container for a fluid dispensing device, the fluid dispensing device comprising a fluid discharge mechanism, the fluid container comprising:
a flexible bag comprising a flexible sidewall, the flexible sidewall extends along a longitudinal direction (z), confines an interior volume to receive the fluid and forms a bag outlet towards a distal end; and
a rigid fastening adapter sealingly engaged with the bag outlet and comprising a hollow outlet shaft in fluid communication with the interior volume;
wherein the rigid fastening adapter comprises a fastening structure for mechanical engagement with a corresponding counter fastening structure of the fluid dispensing device to establish a fluid-tight connection between the fluid discharge mechanism and the outlet shaft.

2. The fluid container according to claim 1, wherein the hollow outlet shaft protrudes outwardly from the interior volume.

3. The fluid container according to claim 1, wherein the rigid fastening adapter comprises a shoulder portion adjacent to the distal end of the flexible bag and wherein the fastening structure is provided on at least one of an outside surface of the shoulder portion and an outside surface of the outlet shaft.

4. The fluid container according to claim 3, wherein the hollow outlet shaft protrudes from the shoulder portion in longitudinal distal direction (z) and wherein the shoulder portion comprises a through recess merging with an interior of the hollow outlet shaft.

5. The fluid container according to claim 3, wherein the shoulder portion comprises a distally facing abutment face to longitudinally abut with the counter fastening structure of the fluid dispensing device.

6. The fluid container according to claim 1, wherein the fastening structure comprises at least one snap element to mechanically engage in a form fitting manner with a complementary-shaped counter snap element of the counter fastening structure.

7. The fluid container according to claim 6, wherein the snap element comprises a beveled flank adjoining a proximally faxing abutment face.

8. The fluid container according to claim 1, further comprising an outer rigid casing sized to accommodate the flexible bag, wherein the outer rigid casing is fixedly connected to the rigid fastening adapter or wherein the rigid fastening adapter is integrated into the outer rigid casing.

9. The fluid container according to claim 8, wherein the outer rigid casing is made of a material or a material composition comprising at least one of a high density polyethylene and a polypropylene.

10. The fluid container according to claim 9 wherein the outer rigid casing comprises a multilayer structure with a first layer made of a high-density polyethylene and a second layer made of a polypropylene.

11. The fluid container according to claim 8, wherein the flexible bag is blow-molded or injection molded into the outer rigid casing, or wherein the flexible bag and the outer rigid casing are co-extruded.

12. The fluid container according to claim 1, wherein the bag outlet is sealingly engaged with the rigid fastening adapter through at least one of a clamping, a bonding, a welding, an extrusion, a blow molding, or an injection molding.

13. The fluid container according to claim 1, wherein an outside surface section of the bag outlet is sealingly engaged with an inside surface of the hollow outlet shaft.

14. The fluid container according to claim 1, wherein the hollow outlet shaft comprises a cylindrical receptacle at a distal end to receive a complementary shaped valve insert of the fluid discharge mechanism.

15. The fluid container according to claim 14, wherein the hollow outlet shaft comprises a tapered seal seat section to sealingly engage with the valve insert.

16. The fluid container according to claim 1, wherein the flexible bag is made of a material or material composition comprising at least one of an ethylene vinyl alcohol, a low density polyethylene and a high density polyethylene.

17. A fluid dispensing device comprising:
a housing to accommodate a fluid container according to claim 1;
an outlet orifice;
a mechanical energy storage coupled to the discharge mechanism, reversibly transferable between a pre-loaded state and an unloaded state and configured to store mechanical energy in the pre-loaded state effective to produce the spray discharging of the fluid;
the discharge mechanism operable for spray discharging multiple doses of the fluid via the outlet orifice; and
the counter fastening structure to mechanically engage with the fastening structure of the rigid fastening adapter of the fluid container to establish the fluid-tight connection between the fluid discharge mechanism and the outlet shaft of the fluid container.

18. The fluid dispensing device according to claim 17, wherein the discharge mechanism comprises a driver operatively coupled to the mechanical energy storage and movable relative to one of the housing and the outlet orifice to effectuate the spray discharging of the fluid, wherein the driver comprises the counter fastening structure to mechanically engage with the rigid fastening adapter of the fluid container.

19. The fluid dispensing device according to claim 17, wherein the discharge mechanism comprises:
an inlet valve in fluid connection with the outlet shaft of the fluid container;
an outlet valve in fluid connection with the outlet orifice;
a dispensing chamber located between the inlet valve and the outlet valve and sized to receive at least one dose of the fluid; and
wherein for effectuating the spray discharging of the fluid a size of the dispensing chamber is reducible by moving at least one of the inlet valve and the outlet valve relative to the other one of the inlet valve and the outlet valve.

20. The fluid dispensing device according to claim 17, further comprising the container filled with the fluid and connected to the outlet orifice in a fluid transferring manner.
